(19) United States
(12) Patent Application Publication
(10) Pub. No.: US 2026/0207452 A1
(43) Pub. Date: Jul. 23, 2026

(54) OIL-CONTINUOUS COSMETIC COMPOSITION

(71) Applicant: Conopco, Inc., d/b/a UNILEVER, Hoboken, NJ (US)

(72) Inventors: Hasiba BEKTO, Bristol, CT (US); Lei HUANG, Trumbull, CT (US)

(73) Assignee: Conopco, Inc., d/b/a UNILEVER, Hoboken, NJ (US)

(21) Appl. No.: 19/140,300

(22) PCT Filed: Nov. 22, 2023
(86) PCT No.: PCT/EP2023/082682

§ 371 Date: Jun. 17, 2025

(30) Foreign Application Priority Data
Dec. 21, 2022 (EP) 22215512.9

(51) Int. Cl. A61K 8/34; A61K 8/06; A61K 8/49; A61K 8/67; A61Q 19/08

(52) U.S. Cl. CPC A61K 8/347; A61K 8/064; A61K 8/4926; A61K 8/671; A61Q 19/08; A61K 2800/34

(57) ABSTRACT

Disclosed herein are personal care compositions. More particularly, disclosed herein is a cosmetic composition containing resorcinol, a functionalized heteroaromatic compound, and retinoic acid precursor in combination with antioxidant, compatible oil, and low HLB, high molecular weight emulsifier to provide for improved stability of the oil-continuous composition.

### FIELD OF THE INVENTION

Disclosed herein are personal care compositions. More particularly, disclosed herein is a cosmetic composition containing resorcinol, a functionalized heteroaromatic compound, and retinoic acid precursor in combination with antioxidant, compatible oil, and low HLB, high molecular weight emulsifier to provide for improved stability of the oil-continuous composition.

### BACKGROUND OF THE INVENTION

Modern definitions of ideal skin presently include youthful and resilient skin having uniform color distribution and smooth texture throughout. However, human skin is subject to deterioration through, for example, dermatological conditions, environmental abuse (wind, air conditioning, sun exposure, pollution, blue light), or chronoaging. Aging individuals increasingly develop facial fine lines, wrinkles, yellowing or sallowness, sagging, hyperpigmentation, age spots and the general signs of aging. Thus, the demand for “anti-aging” or “pre-aging” cosmetic products that address such visible signs has grown greatly.

Cosmetic compositions featuring retinoic acid precursors, such as retinol, have become quite prominent in recent years. Retinol, also known as vitamin A, and many of its ester derivatives and retinoid family members including, for instance, retinyl propionate, can be effective in reducing fine lines and wrinkles, smoothing skin, and improving uneven skin tone among other benefits when applied topically. Notwithstanding the benefits of retinoids, the inclusion of retinoic acid precursors in cosmetic compositions often results in compositions having difficulties with stability.

In addition, many people often search, separately or even simultaneously, for cosmetic products providing even skin tone benefits. The industry is currently marked by an ever-present need for cosmetic even tone agents that have enhanced efficaciousness and stability. Examples of such agents include hydroquinone, arbutin, kojic acid and functionalized heteroaromatic compounds. Without intending to be bound or limited by theory, it is believed that compositions containing a functionalized heteroaromatic compound, (e.g., niacinamide and/or nicotinate) are useful for promoting even skin and skin hydration while reducing skin dullness and the appearance of fine lines, among other benefits. Resorcinols, particularly 4-substituted resorcinols such as 4-hexylresorcinol and 4-ethylresorcinol, provide further examples of agents providing even tone benefits that may be used in topical applications for the skin.

Cosmetic compositions containing even tone agents such as hexylresorcinol have become quite prominent in recent years. However, resorcinol-containing compositions tend to become discolored in response to a variety of factors such as temperature, light, and oxidation. Obtaining cosmetic compositions comprising resorcinols that are color stable is challenging. The issue of color instability in cosmetic compositions is exacerbated when resorcinols are found in the same composition as niacinamide and/or retinoid compounds, resulting in more prominent degradation of color when compared to compositions with each skin benefit agent independently. Thus, the color instability of cosmetic compositions with resorcinols, functionalized heteroaromatic compound and retinoic acid precursors poses even further a challenge to be overcome.

Therefore, the present inventors have recognized a need to develop a cosmetic composition with improved color stability that includes resorcinol, a functionalized heteroaromatic compound and retinoic acid precursor. Disclosed herein is a cosmetic composition having a resorcinol in combination with (a) functionalized heteroaromatic compound, (b) a retinoic acid precursor or (c) a mixture thereof in addition to antioxidant and a compatible oil in a water-in-oil emulsion.

### SUMMARY OF THE INVENTION

Accordingly, in a first aspect, there is provided a cosmetic composition comprising resorcinol, functionalized heteroaromatic compound, retinoic acid precursor, antioxidant, compatible oil, and low HLB, high molecular weight emulsifier, wherein the cosmetic composition is a water-in-oil emulsion.

In a second aspect, there is provided a method of providing an antiaging and/or even tone benefit to the skin comprising the steps of (a) making an oil-continuous cosmetic composition comprising resorcinol, functionalized heteroaromatic compound, and retinoic acid precursor according to the second aspect and (b) applying the cosmetic composition to skin in need of treatment.

In a third aspect, there is provided the cosmetic composition according to the first aspect, wherein the cosmetic composition is substantially free of silicones.

All other aspects of the present cosmetic compositions will become more readily apparent upon considering the detailed description and examples which follow.

Unless explicitly stated otherwise, all ranges described herein are meant to include all ranges subsumed therein. Except in the operating and comparative examples, or where otherwise explicitly indicated, all numbers used in this description indicating amounts, or ratios of materials and/or use thereof are to be understood as modified by the word “about.”

The disclosure of the invention as found herein is to be considered to cover all aspects as found in the claims as being multiply dependent upon each other irrespective of the fact that claim may be found without multiple dependency or redundancy. Unless otherwise specified, numerical ranges expressed in the format “from x to y” are understood to include x and y. In specifying any range of values or amounts, any particular upper value or amount can be associated with any particular lower value or amount. All percentages and ratios contained herein are calculated by weight unless otherwise indicated.

### DETAILED DESCRIPTION OF THE INVENTION

Resorcinol is a monohydroxy phenol compound (i.e., 1,3-dihydroxybenzene) characterized by alcohol groups (—OH) at positions 1 and 3. The chemical structure of resorcinols may be modified, resulting in substituted resorcinols characterized by at least one substituent in the 2, 4, 5 or 6 position. Substituted, as used herein, refers to a compound in which one or more hydrogen atoms are replaced by another atom or group, e.g., substituent. In one aspect, the substituted resorcinol comprises at least one substituent comprising 5 to 11 carbon atoms, or 5 to 9 carbon atoms. In one aspect, the substituted resorcinol has at least one substituent comprised of an alkyl group or unsaturated alkyl group.

In one aspect, the substituted resorcinol is only substituted at position 4 with an alkyl group, e.g., the substituted resorcinol is a 4-substituted alkyl resorcinol, including but not limited to 4-methyl resorcinol, 4-ethyl resorcinol, 4-hexyl resorcinol, 4-cyclohexyl resorcinol, 4-propyl resorcinol, 4-isopropyl resorcinol, 4-butyl resorcinol, 4-pentyl resorcinol, 4-phenylethyl resorcinol, 4-cyclopentyl resorcinol, 4-cyclohexyl resorcinol, 4-heptyl resorcinol, 4-cycloheptyl resorcinol, 4-octyl resorcinol, 4-cyclooctyl resorcinol, 4-nonyl resorcinol, 4-decyl resorcinol, 4-undecyl resorcinol, 4-dodecyl resorcinol and/or combinations or mixtures thereof. Preferably, the substituted resorcinol is 4-ethylresorcinol, 4-hexylresorcinol, and 4-isopropylresorcinol. In one aspect, the substituted resorcinol is only substituted at position 4 being a substituted or unsubstituted 5- or 6-membered ring carbon-based heterocyclic ring containing one or more heteroatoms selected from N, S or O, preferably a substituted 5-membered ring carbon-based heterocyclic ring containing two heteroatoms selected from N or S, e.g., isobutylamido thiazolyl resorcinol or thiamidol. The composition comprises 0.001 to 10%, or 0.01 to 5%, or 0.025 to 2.5%, or 0.05 to 2%, or 0.1 to 1% resorcinol by weight of the cosmetic composition, including all ranges subsumed therein. In yet another embodiment, the resorcinol is 4-hexylresorcinol, 4-ethylresorcinol, or mixtures thereof.

The cosmetic composition disclosed herein comprises a retinoic acid precursor, which typically includes retinyl esters, retinol, retinal, or mixtures thereof. The terms “retinoid” and “retinoic acid precursor,” as used herein, refers to all natural and/or synthetic analogues of vitamin A or derivatives thereof having similar biological activity as vitamin A in human skin. In one embodiment, the retinoic acid precursor comprises retinal, retinyl ester, or mixtures thereof. Examples of retinyl esters desirable for use include but are not limited to: retinyl palmitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl taurate, retinyl tridecanoate, retinyl myristate, retinyl pentadecanoate, retinyl heptadeconoate, retinyl stearate, retinyl isostearate, retinyl nonadecanoate, retinyl arachidonate, retinyl behenate, retinyl linoleate, and retinyl oleate. In an embodiment, the ester is selected from retinyl palmitate, retinyl acetate, retinyl propionate, and mixtures thereof. In another embodiment, the retinoic acid precursor includes retinyl linoleate, retinyl oleate, and mixtures thereof. In yet another embodiment, the retinoic acid precursor is retinyl propionate, retinol, or mixtures thereof. Retinoic acid precursor is employed in an amount of 0.001 to 5%, or 0.01 to 2.5%, or 0.025 to 2%, or 0.05 to 1%, or 0.1 to 0.5% by weight of the cosmetic composition, including all ranges subsumed therein.

Cosmetic compositions disclosed herein include, in addition to resorcinol and retinoic acid precursor, a functionalized heteroaromatic compound. It is desirable to use a carboxylic acid functionalized heteroaromatic compound to deliver the skin benefits as described herein. The carboxylic acid functionalized heteroaromatic compound may be any cosmetically acceptable nicotinic acid, picolinic acid, or derivative and mixtures thereof, or nicotinic acid, niacinamide, nicotinate, picolinic acid, picolinamide, and mixtures thereof, or niacinamide, picolinamide, and mixtures thereof. The functionalized heteroaromatic compound is typically present in an amount of 0.001 to 10%, or 0.025 to 8%, or 0.05 to 6%, or 0.1 to 5%, or 0.5 to 4% by weight of the cosmetic composition, including all ranges subsumed therein.

Now it has surprisingly been determined that use of a compatible oil in compositions with resorcinol, retinoic acid precursor and a functionalized heteroaromatic compound can contribute to improved color stability. The term “compatible” as used herein refers to an oil possessing a Hansen total solubility parameter value (δₜ) within the range of 15 to 23, or 16 to 22, or 17 to 21. Hansen total solubility parameters is determined based on three Hansen parameters, namely δₜ (defines energy from dispersion forces between molecules), δₚ (defines energy from dipolar intermolecular forces between molecules), and δₙ (defines energy from hydrogen bonds between molecules). δₜ values are obtained according to industry references such as the CRC Handbook of Solubility Parameters and other Cohesion Parameters, Second Edition by Allan F. M. Barton or software such as the Hansen Solubility Parameters in Practice (HSPIP). Illustrative examples of compatible oils that can be used in the compositions disclosed herein include caprylic/capric triglycerides (δₜ=18.9), ethylhexyl salicylate (δₜ=18.9), isodecyl neopentanoate (δₜ=15.8), isopropyl myristate (δₜ=17.5), isopropyl palmitate (δₜ=17.2), octocrylene (δₜ=18.6), octylmethoxycinnamate (OMC) (δₜ=18.9), and combinations thereof. Compatible oils are employed in an amount of 10 to 40%, or 12 to 30%, or 14 to 25%, or 15 to 20% by weight of the cosmetic composition, including all ranges subsumed therein.

It is within the scope of the cosmetic compositions to further include one or more antioxidants, which may be natural, naturally derived, or synthetic antioxidants, as it has also unexpectedly been found to enhance color stability in such cosmetic compositions. Antioxidants may be any antioxidant desirable for use in cosmetic and/or pharmaceutical compositions. Illustrative but nonlimiting examples of such antioxidants comprise alpha hydroxy acids (e.g., citric acid, lactic acid, glycolic acid and mandelic acid), amino acids and derivatives thereof, beta-hydroxy acids (e.g., propanoic acid and salicylic acid), butylated hydroxytoluene (BHT), carotenes, carotenoids, co-enzyme Q10, didodecyl 3,3′ thiodipropionate, glutathione, imidazole and derivatives thereof, octadecyl di-t-butyl-4-hydroxyhydrocinnamate, pentaerythrityl tetra-di-t-butyl hydroxyhydrocinnamate, peptides and derivatives thereof, plant extracts (e.g., Rosmarinus officinalis (rosemary) leaf extract and Camellia sinesis (green tea) leaf extract), polyhydroxy acids (e.g., gluconic acid, gluconolactone and lactobionic acid), polyphenols (e.g., anthocyanin, ellagic acid, flavonoid, tannin), and resveratrol.

Another class that may be used in the disclosed cosmetic composition as antioxidants includes vitamins. Illustrative vitamins include, but are not limited to, vitamin B₂, vitamin B₅ (panthenol), vitamin B₆, vitamin C, vitamin E, vitamin F, vitamin K, folic acid, and biotin. Derivatives of the vitamins may also be employed. For instance, vitamin C derivatives include ascorbyl tetraisopalmitate, magnesium ascorbyl phosphate and ascorbyl glycoside. Derivatives of vitamin E include tocopheryl acetate, tocopheryl palmitate and tocopheryl linoleate.

It is preferred that the antioxidant comprises BHT, didodecyl 3,3′ thiodipropionate, octadecyl di-t-butyl-4-hydroxyhydrocinnamate, pentaerythrityl tetra-di-t-butyl hydroxyhydrocinnamate, polyphenols, or a mixture thereof. The total weight percent of antioxidants present in the cosmetic compositions disclosed herein ranges from 0.01 to 4%, or 0.02 to 3%, or 0.05 to 2% by weight of the cosmetic composition, including all ranges subsumed therein. The minimum total weight percent of antioxidants present in the cosmetic compositions is 0.1%, or 0.15%, or 0.20% by weight based on the total weight of the composition.

Now it has surprisingly been determined that use of an emulsion, or a water-in-oil (i.e., oil-continuous) emulsion, or a silicone-free water-in-oil emulsion, in cosmetic compositions with resorcinol, retinoic acid precursor, and a functionalized heteroaromatic compound can contribute to enhanced color stability of the overall composition.

Nonionic emulsifiers desirable for inclusion in the cosmetic composition disclosed herein are low HLB surfactants with an HLB value less than or equal to 8, or an HLB less than or equal to 7, or an HLB less than or equal to 6, or an HLB ranging from 2 to 8, or an HLB ranging from 2 to 7, or an HLB ranging from 3 to 6. In one embodiment, the nonionic emulsifier has a number average molecular weight greater than 500, or greater than 600, or greater than 700. In another embodiment, the nonionic emulsifier has a number average molecular weight ranging from 500 to 5000 Da, or 800 to 4000 Da, or 1000 to 3000 Da, or 1500 to 2500 Da. The nonionic emulsifier is typically present in an amount of 0.1 to 10%, or 0.5 to 7%, or 0.75 to 5%, or 1 to 3% by weight of the cosmetic composition, including all ranges subsumed therein.

Nonionic emulsifiers suitable for inclusion into the disclosed cosmetic composition are nonionic copolymeric emulsifiers with an HLB less than or equal to 8, or less than or equal to 7, or less than or equal to 6, and are selected from the group consisting of block copolymer of type ABA, polysiloxane polyalkyl polyether copolymers and mixtures thereof. The nonionic copolymeric emulsifiers described below are suitable for use in the disclosed cosmetic composition, as long as they are selected also to have HLB less than or equal to 8.

One type of ABA block copolymer is that having the general formula A-COO—B—OOC-A, in which B is the divalent residue of a water-soluble polyalkylene glycol and A is the residue of an oil-soluble complex monocarboxylic acid i.e., a fatty acid, with a number average molecular weight of at least 500 Da, or at least 800 Da. These complex monocarboxylic acids may be represented by the general formula:

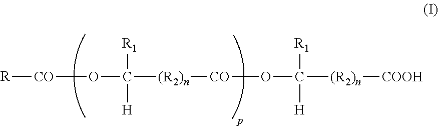

in which
R is hydrogen or a monovalenthydrocarbon or substituted hydrocarbon group;
R₁ is hydrogen or a monovalent C₁ to C₂₄ hydrocarbon group;
R₂ is a divalent C₁ to C₂₄ hydrocarbon group;
n is zero or 1;
p is an integer from zero to 200.

The units between the brackets in general formula I may be all the same or they may differ in respect of R₁, R₂, and n. The quantity p will not normally have the same unique value for all molecules of the complex acid but will be statistically distributed about an average value lying within the range stated, as is commonplace in polymeric materials. Polymeric component B has a number average molecular weight of at least 500 Da, or at least 800 Da, or 800 to 4000 Da, or 1000 to 3000 Da, or 1500 to 2500 Da, and is the divalent residue of a water-soluble polyalkylene glycol having the general formula

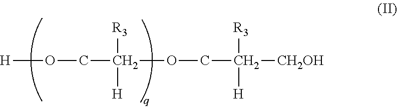

wherein
R₃ is hydrogen or a C₁ to C₃ alkyl group;
q is an integer from 10 up to 500.

The repetitive units in general formula II again may all be the same or may differ in R₃, and the quantity q may vary about an average value.

The hydrocarbons R, R₁, and R₂ may be linear or branched.

In one aspect, in the block copolymers of formula A-COO—B—OOC-A, component B is derived from polyethylene glycol and components A are derived from stearic acid, for example, polyhydroxystearic acid, or from poly(12-hydroxystearic acid).

Thus, R may be a straight chain C₁₇H₃₅-group derived from stearic acid, and the unit containing R₁ and R₂ may be derived from 12-hydroxystearic acid.

p, in this case, preferably has a value of at least 2.

Preferably q may have a value between 20 and 60, or above 23, or 23 to 50. The weight ratio of the combined components A to the component B may vary widely, and typically will lie in the range from 9:1 to 1:9.

In one embodiment, the low HLB nonionic copolymeric emulsifier used according to the cosmetic compositions disclosed herein is Cithrol™ DPHS, a PEG-30 dipolyhydroxystearate. Another, similar, nonionic emulsifier for use in the invention is Atlox™ 4912. Both Cithrol™ DPHS and Atlox™ 4912 are block copolymers (A-B-A) of polyethylene glycol and polyhydroxystearic acid with a number average molecular weight of approximately 5000 Da commercially available from Croda International PLC. The preferred copolymeric surfactant for use herein comprises PEG-30 dipolyhydroxystearate (wherein 30 here designates the number of polyethylene glycol repeat units).

Another type of ABA block copolymer is that consisting of propylene oxide (PO) and ethylene oxide (EO) blocks otherwise known as Pluronics. Representative of this class are the polyether surfactants comprising block polymers of two or more different kinds of oxyalkylene repeat units, the ratio of which determining the HLB of the surfactant. The straight chain polyether surfactants are available from BASF under the registered trademark “Pluronic (BASF).” For convenience purposes, the straight chain surfactants employed in the composition disclosed herein will be referred to as Pluronic generally, and with a numerical suffix to identify a particular grade of material.

Pluronic are block copolymers consisting of propylene oxide (PO) and ethylene oxide (EO) blocks—specifically, they are poly(A-oxyethylene-B-oxypropylene-A-oxyethylene)triblock copolymers. Suitable straight chain polyether surfactants having HLB value less than or equal to 8, or less than or equal to 7, or less than or equal to 6 include for example but are not limited to:
Pluronic™ L31 (BASF) having a HLB of 5 and average molecular weight (AMW) of 1100;
Pluronic™ L42 (BASF) having a HLB of 8 and average molecular weight (AMW) of 1630;
Pluronic™ L61 (BASF) having a HLB of 3 and average molecular weight (AMW) of 2000;
Pluronic™ L62 (BASF) having a HLB of 7 and average molecular weight (AMW) of 2500;
Pluronic™ L72 (BASF) having a HLB of 7 and average molecular weight (AMW) of 2750;
Pluronic™ L81 (BASF) having a HLB of 2 and average molecular weight (AMW) of 2750;
Pluronic™ L92 (BASF) having a HLB of 6 and average molecular weight (AMW) of 3650;
Pluronic™ L101 (BASF) having a HLB of 1 and average molecular weight (AMW) of 3800;
Pluronic™ L121 (BASF) having a HLB of 1 and average molecular weight (AMW) of 4400;
Pluronic™ L122 (BASF) having a HLB of 4 and average molecular weight (AMW) of 5000;
Pluronic™ P123 (BASF) having a HLB of 8 and average molecular weight (AMW) of 5750.

Most preferred Pluronic surfactants are Pluronic™ L61, L62, L72, L81, L92, L101, L121, L122, L123.

Nonionic, low HLB emulsifiers desirable for inclusion into the disclosed cosmetic composition also comprise polyglycerol esters with an HLB value of less than or equal to 8, or an HLB less than or equal to 7, or an HLB less than or equal to 6, and mixtures of such polyglyceryol ester emulsifiers may also be desirable. Illustrative and nonlimiting examples of such polyglycerol esters include polyglyceryl-3 stearate, polyglyceryl-4 stearate, polyglyceryl-6 distearate, and polyglyceryl-10 decaoleate. Other desirable nonionic, low HLB emulsifiers comprise hexaglyceryl polyricinoleate.

In one embodiment, the nonionic emulsifier comprises PEG-30 dipolyhydroxystearate, cetyl PEG/PPG-10/1 dimethicone, polyglyceryl-6 polyricinoleat, polyglyceryl-3 diisostearate, or a mixture thereof.

Other nonionic, low HLB emulsifiers having HLB values of less than or equal to 8, or an HLB less than or equal to 7, or an HLB less than or equal to 6, may be optionally included in the cosmetic compositions disclosed herein. Illustrative but nonlimiting examples of such optional emulsifiers include polysiloxane polyalkyl polyether copolymers, i.e., silicone glycol surfactants which are also known as dimethicone copolyols. The silicone glycol surfactant is present in amounts of 0.1 to 5%, or 0.25 to 3%, or 0.5 to 2% by weight of the cosmetic composition. Such silicone glycol surfactants include high molecular weight polymers of dimethyl polysiloxane with polyoxyethylene and/or polyoxypropylene side chains, having a number average molecular weight of from 500 to 5,000 Da.

Nonionic sugar surfactants selected from the group consisting of sugar fatty acid esters are also suitable for optional inclusion into the disclosed cosmetic composition as other nonionic, low HLB emulsifiers. The nonionic emulsifiers described below are suitable for use in the disclosed cosmetic composition, as long as they are selected also to have a low HLB and a high molecular weight.

Esters of fatty acid and sugar include esters or mixtures of esters of linear or branched and saturated or unsaturated C₁₂ to C₂₂ fatty acids and of sucrose, maltose, glucose, fructose, mannose, galactose, arabinose, xylose, lactose, trehalose, or methylglucose. These esters may be chosen from mono-, di-, tri- and tetraesters, polyesters, and their mixtures. The C₁₂ to C₂₂ fatty acids includes C₁₂, C₁₃, C₁₄, C₁₅, C₁₆, C₁₇, C₁₈, C₁₉, C₂₀, C₂₁, and C₂₂, in any subrange or combination. In one embodiment, these esters are chosen from stearates, behenates, cocoates, arachidonates, palmitates, myristates, laurates, carprates, oleates, laurates, and their mixtures. Mixtures of these derivatives are possible. If included, sucrose esters are preferably used, including sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose polyesters, such as sucrose pentaoleate, hexaoleate, heptaoleate or octooleate, and mixed esters, such as sucrose palmitate/stearate, and mixtures thereof. In one aspect, the sucrose ester comprises sucrose distearate, sucrose tristearate, sucrose polystearate, or mixtures thereof.

Preferable esters or mixtures of esters of fatty acid and of sucrose include sucrose distearate as made commercially available by Croda as Crodesta™ F10 and sucrose polystearate as made commercially available by MMP, Inc. as Sisterna® SP10-C.

Other preferable esters or mixtures of esters of fatty acid and of methylglucose include the distearate of methylglucose and of polyglycerol-3 made commercially available by Evonik Corporation under the name of TEGO® Care 450. Still other desirable esters include the oleate, isostearate, or sesquistearate of methyl glucose. These are sold under the tradename Glucate™ DO, Isolan® IS, Glucate™ SS. Glucate™ DO and SS are available from The Lubrizol Corporation and Isolan® IS from Evonik Corporation. Mention may also be made of glucose or maltose monoesters, such as methyl O-hexadecanoyl-6-D-glucoside and (lacuna) O-hexadecanoyl-6-D-maltose.

Other sugar fatty acid ester derivatives which can be used as nonionic emulsifiers in the disclosed cosmetic composition include sugar fatty esters which are optionally oxyalkylenated (oxyethylenated and/or oxypropylenated) or polyglycerolated. Preferable oxyethylenated esters of fatty acid and of sugar include oxyethylenated (20 EO) methylglucose sesquistearate, such as the product sold under the tradename Glucamate™ SSE-20, by The Lubrizol Corporation.

Color stability is determined through the use of LabScan XE equipment (Hunter Associates Laboratory, Inc. Reston, Virginia). LabScan XE measures color data. Samples at different storage stages housed in vials or in measurement cells are loaded onto the LabScan XE measurement port. By following the measurement procedure specified in the associated instrument menu and computer software, colors (a*, b* and L*) are measured and calculated to result in a color change data (ΔE) value. For reference, a ΔE of approximately three is the threshold where one may visibly detect a color change from the original sample. Remains color stable, as used herein, describes a cosmetic composition, without masking agents, that possesses a ΔE of 5.5 or less based on L*, a*, and b* color differences taken on a Hunter Lab spectrophotometer at 4 weeks, or 8 weeks at 45° C. Generally, ΔE≤5.5 at 4 weeks at 45° C. is desirable whereas ΔE>5.5 is undesirable. In one embodiment, ΔE≤9.5 at 8 weeks at 45° C. is desirable and ΔE>9.5 is undesirable.

It has also been unexpectedly discovered that other compositions made with the present cosmetic compositions provide good stability and color stability for the composition itself, as well as anti-aging and even tone benefits when applied to the skin.

Cosmetic compositions disclosed herein may also comprise a cosmetically acceptable vehicle to act as a diluent, dispersant or carrier for the skin benefit agents in the composition, so as to facilitate its distribution when the composition is applied to the skin. This cosmetically acceptable vehicle may be aqueous, anhydrous, or an emulsion. Oily carriers in the presence of water and an emulsifier will form emulsion systems as carriers. In one embodiment, the composition is an emulsion, especially a water-in-oil emulsion. For the avoidance of doubt, water-in-oil emulsions are also known as an oil-continuous emulsions. The cosmetic compositions ordinarily will be in but are not limited to cream or lotion form.

Suitable carriers employed include water, sea water, rosewater (e.g., Rosa Damascena flower Water), tea (e.g., Camellia Sinensis Leaf Water), Aloe Barbadensis (aloe vera) leaf juice, witch hazel (e.g., Hamamelis Virginiana extract), lavender water (e.g., Lavendula Angustifolia flower water), grape water (e.g., Vitis Vinifera fruit water and Vitis Vinifera juice). In the most preferred embodiment, water is the carrier. Amounts of water can be 5 to 85%, or 15 to 80%, or 30 to 75% by weight based on the weight of the cosmetic composition. The cosmetically acceptable vehicle can, in the absence of other cosmetic adjuncts, form the balance of the composition. Besides water, desirable carrier classes include, but are not limited to, polyhydric alcohols, silicones, fatty acids, hydrocarbons, triglycerides, waxes, and thickening agents.

Conventional humectants, generally of the polyhydric alcohol-type materials, can be optionally included in the cosmetic compositions disclosed herein. Typical polyhydric alcohols include glycerol (i.e., glycerine or glycerin), propylene glycol, propanediol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol, and mixtures thereof. A preferred embodiment includes glycerin, propylene glycol, or a mixture thereof. The amount of humectant employed can be from 0.1 to 25%, or 0.5 to 20%, or 1 to 15% by weight of the cosmetic composition.

The disclosed cosmetic composition may optionally comprise silicones. Silicones may be categorized into the volatile and nonvolatile variety. Amounts can be, for example, 0.01 to 5%, or 0.1 to 3% by weight of the composition. The term “volatile” as used herein refers to those materials which have a measurable vapor pressure at ambient temperature. Volatile silicone oils may be chosen from cyclic (cyclomethicone) or linear polydimethylsiloxanes containing from 3 to 9, or from 4 to 5, silicon atoms.

Nonvolatile silicones useful in this composition include polyalkyl siloxanes, polyalkylaryl siloxanes and polyether siloxane copolymers. The essentially nonvolatile polyalkyl siloxanes useful herein include, for example, polydimethyl siloxanes with viscosities from 5×10⁻⁶ to 0.1 m²/s at 25° C. Emulsifying and non-emulsifying silicone elastomers are also suitable for use in the cosmetic compositions disclosed herein.

In one aspect, the cosmetic composition is substantially free of silicone. “Substantially free of” as used herein, is intended to mean comprising less than 5% by weight, or less than 2% by weight, or less than 0.5% by weight, or less than 0.1% by weight, or 0% by weight of the composition. In still another aspect, the cosmetic composition does not contain any (i.e., 0% by weight) silicone.

Suitable carrier may also be fatty acids, wax and wax esters, triglycerides, or mixtures thereof. In one embodiment, the carrier may comprise at least 50% by weight, or at least 75% by weight, or entirely (i.e., 100% by weight) of an aforementioned compatible oil. In another embodiment, the carrier comprises at least 50% by weight, or at least 75% by weight, or entirely of caprylic/capric triglyceride. In yet another embodiment, the carrier comprises at least 50% by weight, or at least 75% by weight, or entirely of isopropyl myristate. In still another embodiment, the carrier comprises at least 50% by weight, or at least 75% by weight, or entirely of isodecyl neopentanoate.

Thickening agent may be optionally desirable for inclusion in the present cosmetic compositions. Useful thickening agents include polysaccharides, which comprise of starches, natural/synthetic gums, and cellulosics. Desirable starches include tapioca starch, corn starch, potato starch, aluminum starch octenylsuccinate, and sodium hydroxypropyl starch phosphate. Desirable gums include xanthan, sclerotium, pectin, karaya, arabic, agar, guar, carrageenan, alginate, and combinations thereof. Desirable cellulosics include hydroxypropyl cellulose, hydroxypropyl methylcellulose, ethylcellulose, and sodium carboxy methylcellulose. Thickening agent may be present in the cosmetic composition disclosed herein in amounts ranging from 0.001 to 3%, or 0.1 to 2%, or 0.2 to 1% by weight of the composition. In one embodiment, the cosmetic composition is substantially free of thickening agent. In another embodiment, the cosmetic composition comprises no (0% by weight) thickening agent.

The cosmetic compositions disclosed herein are optionally substantially free of synthetic polymer microparticles (i.e., microplastic particles). As used herein, synthetic polymer microparticles refer to polymers that are solid and which either are contained in particles and constitute at least 1% by weight of those particles, or build a continuous surface coating on particles, where at least 1% by weight of those particles fulfill either of the conditions: (a) all dimensions of the particles are equal to or less than 15 mm and their length to diameter ratio is greater than 3; and/or (b) the length of the particles is equal to or less than 15 mm and their length to diameter ratio is greater than 3. The definition for synthetic polymer microparticles is consistent with the definition proposed by the European Chemical Agency (ECHA). Illustrative but nonlimiting examples of such as such synthetic polymer microparticles include acrylate-based polymers acrylamide/sodium acrylate copolymer, acrylates/beheneth-25 methacrylate copolymer, acrylates/C₁₀₋₃₀ alkyl acrylate crosspolymer, acrylates/dimethicone copolymer, carbomer, and sodium acrylate/sodium acryloyldimethyl taurate copolymer. In one embodiment, the cosmetic composition is substantially free of acrylate-based polymers, or comprises no (0% by weight) acrylate-based polymers.

One of ordinary skill in the art would be adept to identify any need of neutralization for the cosmetic composition disclosed herein. Any pH modifier known in the field acceptable for use in personal care and/or pharmaceutical products can be employed to result in a cosmetic composition possessing a skin-acceptable pH, typically within the range of 4 to 8. In a preferred embodiment, the pH of the cosmetic composition is 4.25 to 7.5, and most preferably, 5 to 7. Traditional buffers or pH modifiers include common additives such as sodium hydroxide, potassium hydroxide, hydrochloric acid, citric acid, triethanolamine and aminomethyl propanol. Viscosity of the cosmetic composition disclosed herein is preferably 1,000 to 120,000 centipoise (cps), and, more preferably, 5,000 to 80,000 cps, taken under conditions of ambient temperature and a shear rate of 1 s⁻¹ with a strain controlled parallel plate rheometer made commercially available from suppliers like T.A. Instruments under the Discovery name. Alternatively, viscosity can also be measured using a Brookfield Viscometer (speed at 20 rpm, spindle 5, helipath off, for one (1) minute at ambient temperature). The cosmetic composition can be formulated as a serum having a viscosity of 1,000 to 4,000 millipascal-second (mPas), a lotion having a viscosity of 4,000 to 10,000 mPas, a fluid cream having a viscosity of 10,000 to 20,000 mPas or a cream having a viscosity of 20,000 to 100,000 mPas or above.

Fragrances, skin benefit agents, fixatives and abrasives may optionally be included in cosmetic compositions disclosed herein. Skin benefit agents include but are not limited to opacifiers, colorants, humectants, emollients, occlusive agents, plant extracts, optical agents, even tone agents, anti-inflammatory agents, anti-acne agents, sunscreens, photostabilizers, wrinkle-reducing agents, desquamation promoters, exfoliating agents, mixtures thereof or the like. Each of these substances may be present in an amount of 0.05 to 5%, or 0.1 to 3% by weight of the cosmetic composition.

In one aspect, the cosmetic composition is biodegradable, or 90% of the composition is biodegradable, or 95% biodegradable, or 97% biodegradable, or 99 to 100% biodegradable.

In another aspect, the cosmetic composition is substantially free of sulfates. In still another aspect, the cosmetic composition does not contain any (i.e., 0% by weight) sulfate.

Retinoid boosters may also be useful ingredients to incorporate for use in the compositions disclosed herein, such as fatty acids and derivatives thereof. As described in several U.S. Patents to Granger et al. (U.S. Pat. Nos. 5,759,556, 5,756,109, 5,747,051, 5,716,627, 5,811,110, 5,536,740, 5,747,051, 5,599,548, 5,955,092, 5,885,595, 5,759,556, 5,693,330, 7,959,913, 8,226,933 and 8,409,550), the term “retinoid boosters” is used to refer collectively to compounds that have been determined to enhance the enzymatic conversion of retinyl esters and retinol to retinoic acid as part of the natural retinol metabolic process in the skin's epidermis. The boosters alone or in combination with other booster compounds help to enhance efficacy of a retinoic acid precursor. The term “fatty” refers to carbon chain lengths ranging from 10 to 30 carbon atoms.

Illustrative of this category are pelargonic, lauric, myristic, palmitic, stearic, isostearic, hydroxystearic, oleic, linoleic, ricinoleic, arachidic, behenic, erucic acids and combinations thereof. Preferred derivatives of such fatty acids include fatty alcohols, such as cetyl, lauryl, myristyl, palmitic, cetearyl, stearyl and oleyl alcohols; fatty esters, such as 2-ethyl-hexyl myristate, isopropyl stearate and isostearyl palmitate, triisopropyl trilinoleate and trilauryl citrate, lauryl palmitate, myristyl lactate, oleyl eurcate and stearyl oleate, coco-caprylate/caprate (a blend of coco-caprylate and coco-caprate), propylene glycol myristyl ether acetate, diisopropyl adipate and cetyl octanoate; and fatty amides, such as cocamide monoethanolamide (CMEA), castor oil monoethanolamide, linoleoyl monoethanolamide (LAMEA), palmitamide monoethanolamide, and linoleamide diethanolamide.

Other illustrative but nonlimiting examples of such booster compounds include carotenoids, flavonoids, cyclic and non-cyclic fragrances, antimicotics (e.g., bifonazole, climbazole, clotrimazole, econazole, ketoconazole, miconazole), phospholipid analogues, ureas, phosphatiylethanolamine, phosphatidylcholine, sphingomyelin, natural colorants (e.g., coumarin), quinolines, isoquinolines, metyrapone, and mixtures thereof. In an embodiment, fatty acids, fatty alcohols, fatty amides, climbazole, or a mixture thereof may be used as retinoid boosters. In a particularly preferred embodiment, 12-hydroxystearic acid, cetyl alcohol, cetearyl alcohol, CMEA, LAMEA, climbazole, or a mixture thereof is used as retinoid boosters. When used, 0.01 to 5% by weight of a retinoid booster compound is present in the composition.

It is within the scope of the cosmetic compositions to optionally include sunscreens and photostabilizers. The sunscreens and photostabilizers that may be used in the cosmetic compositions include such materials as phenylbenzimidazole sulfonic acid (Ensulizole), ethylhexyl p-methoxycinnamate, available as PARSOL MCX®, Avobenzene (butyl methoxydibenzoylmethane), available as PARSOL 1789® and benzophenone-3, also known as oxybenzone. Inorganic sunscreen actives may be employed such as microfine titanium dioxide (preferably, with a particle diameter of less than 150 nanometers (nm), or less than 100 nm) and zinc oxide may be used, polyethylene and various other polymers are also desirable sunscreens. Other sunscreens and photostabilizers desirable for use include p-aminobenzoic acid (PABA), benzophenone-1, benzophenone-2, benzophenone-4, benzophenone-6, benzophenone-8, benzophenone-9, benzophenone-12, bisoctriazole, bisethylhexyloxyphenol methoxyphenyl triazine, bisdisulizole disodium, diometriazole trisiloxane, 2-ethoxyethyl p-methoxy cinnamate, homomethyl salicylate, iscotrizinol, isopentenyl-4-methoxycinnamate, menthyl anthranilate, polysilicone-15, triethanolamine salicylate, terephthalylidene dicamphor sulfonic acid, octyldimethyl-PABA, octyltriazone, mixtures thereof or the like. Amounts of the sunscreen or photostabilizing agents, when present, may generally be present in an amount of 0.1 to 20%, or 0.5 to 15%, or 0.75 to 10% by weight of the cosmetic composition.

Other additional optional skin benefit agents desirable for use include minerals and skin nutrients such as milk; magnesium, calcium, copper, zinc and other metallic components; kojic acid; hydroquinone and arbutin; saccharide isomerate; undecylenoyl phenylalanine; sphingolipids, phospholipids (e.g., phytosphingosine), ceramides (e.g., Ceramide 1, Ceramide 3, Ceramide 3B and Ceramide 6) and pseudoceramides; allantoin; pyroglutamic acid (PCA) salt derivatives including zinc PCA and sodium PCA; petroselinic acid; conjugated linoleic acid; octadecanoic acid; hyaluronic acid and its salt derivatives; and mixtures thereof and the like. Such skin benefit agents, when used, collectively make up 0.001 to 12% by weight of the cosmetic composition.

A wide selection of botanical extracts may optionally be included in the cosmetic compositions. The extracts may either be soluble in water or oil, carried in a solvent that is hydrophilic or hydrophobic, respectively. In a preferred embodiment, water or ethanol are the extract solvents. Illustrative examples include those extracted from green tea, yarrow, chamomile, licorice, aloe vera, citrus unshui, willow bark, alfalfa, algae, witch hazel, sage, thyme, and rosemary, as well as oils such as those derived from sea buckthorn, moringa, argan, avocado, calendula, algal, and marula. Soy extracts may also be used. Such extracts, when used, are preferably employed in collective amounts of 0.001 to 12% by weight of the cosmetic composition.

Another optional additive desirable for use includes hemp oil with 2.5 to 25% by weight cannabigerol and/or cannabidiol at from 0.5 to 10 percent by weight. When used, such oil makes up 0.0001 to 12%, or 0.01 to 5% by weight of the cosmetic composition.

Optionally, one or more coloring agents may also be included in the cosmetic compositions disclosed herein. Coloring agents may help mask discoloration and/or reduce perceivable color change during storage stability. Coloring agents include dyes or pigments of natural or synthetic origin. A dye selected for use may be organic or inorganic and water-soluble or oil-soluble at ambient temperature and atmospheric pressure. Illustrative and nonlimiting examples of water-soluble dyes include, for example, D&C Yellow 8, D&C Yellow 10, D&C Orange 4, D&C Red 6, D&C Red 22, D&C Red 28, D&C Red 33, D&C Green 5 or methylene blue. Oil-soluble dyes include, for example, D&C Red 17, D&C Green 6, β-carotene, D&C Violet 2, D&C Yellow 11, D&C Orange 5 and quinoline yellow. Pigments desirable for use are specially chosen from the mineral pigments, organic pigments, lakes, and mixtures thereof as known in the art. Illustrative but nonlimiting examples include mineral pigments such as ochres (yellow ochre, red ochre, brown ochre, iron hydroxide), metal oxides (zirconium oxide, iron oxide, cerium oxide, chromium oxide), manganese violet, ultramarine blue, chromium hydrate and ferric blue, or metal powders (aluminum, bronze or copper powder); organic pigments such as nitroso, nitro, azo, phthalocyanine, diazine, xanthene, pyrene, quinoline, anthraquinone, triphenylmethane, fluorane, quinacridone, metal complex, isoindolinone, isoindoline, perinone, perylene, diketopyrrolopyrrole, indigo, thioindigo, dioxazine, triphenylmethane and quinophthalone compounds; and lakes (particularly those deriving from calcium, barium, aluminum and strontium salts) such as D&C Red No. 6 Barium Lake, D&C Red No. 7 Calcium Lake, D&C Red No. 27 Aluminum Lake, D&C Red No. 28 Aluminum Lake, D&C Red No. 33 Aluminum Lake, FD&C Red No. 40 Aluminum Lake, FD&C Yellow No. 5 Aluminum Lake, FD&C Yellow No. 6 Aluminum Lake, D&C Yellow No. Aluminum Lake, D&C Orange No. 5 Aluminum Lake and F&D Blue No. 1 Aluminum Lake. Nacres, such as natural mica coated with a metal oxide, bismuth oxychloride or a natural pigment, are also desirable to be included in the cosmetic compositions disclosed herein. Other coloring agents desirable for use include chalk, activated charcoal, and carbon black. Cosmetic compositions comprise 0.01 to 5%, or 0.1 to 3%, or 0.5 to 1.5% by weight of the coloring agent, when used, relative to the weight of the cosmetic composition.

Preservatives can be incorporated into the present cosmetic compositions as desired to protect against the growth of potentially harmful microorganisms. Cosmetic chemists are familiar with appropriate preservatives and routinely choose them to satisfy preservative tests and product stability tests. Preservative systems should be selected having regard for the use of the composition and possible incompatibilities between the preservatives and other ingredients in the formulation. Exemplary examples of preservatives for cosmetic compositions disclosed herein include, without limitations, iodopropynyl butyl carbamate (IPBC), phenoxyethanol, ethylhexylglycerine, 1,2-octanediol, methyl paraben, propyl paraben, imidazolidinyl urea, sodium dehydroacetate, propanediol, alkyl esters of para-hydroxybenzoic acid, organic acids and salts thereof, hydroxyacetophenone, DMDM hydantoin derivatives, climbazole, propionate salts, a variety of quaternary ammonium compounds, and combinations thereof. Preservatives may be employed in amounts of 0.01 to 2% by weight of the composition.

Traditional fragrance components like eugenol, coumarin, linalyl acetate, citronellal, iris concentrate, terpinyl acetate, terpineol, thymol, pinenes (e.g., alpha and beta pinene) and citronellol may optionally be added to the cosmetic composition as well.

If employed, the traditional preservatives, vicinal diol and/or fragrance component will not make up more than 5%, or not more than 2%, or from 0.2 to 0.85% by weight of the cosmetic composition disclosed herein. In one aspect, from 0.2 to 0.8% by weight optional preservative, vicinal diol and/or fragrance component is used, based on total weight of the cosmetic composition. In another aspect, the cosmetic composition may comprise of a preservative system that is formaldehyde-free, paraben-free, or both.

When making the present cosmetic composition, the desired ingredients are mixed via conventional methods. If applicable, the oil phase and water phases are separately mixed using standard mixers and mixing blades and heated to a temperature usually from 22 to 85° C. under atmospheric pressure. Upon reaching the desired temperature, the oil phase is added to the water phase to create an emulsion. Other ingredients in the cosmetic composition are subsequently added and mixed in no particular order.

The cosmetic composition disclosed herein is a composition suitable for topical application to human skin, including leave-on and wash-off products. Preferably, the term encompasses a fluid liquid, and particularly a moisturizer rather than a make-up product. Most preferred are leave-on compositions. The term “leave-on” as used with reference to compositions herein means a composition that is applied to or rubbed on the skin and left thereon.

Many types of packaging can be used to store and deliver the present cosmetic composition. The selection of packaging is dependent upon the personal care end-use and the viscosity of the composition itself. As an example, leave-on lotions and creams for skin typically employ plastic containers with an opening at a dispense end covered by an appropriate closure. Conventional closures include flip-top hinged lids, screw-caps and non-aerosol pumps. As another example, appropriate packaging to be used for antiperspirants, deodorants and depilatories include a container with a roller-ball applicator on a dispensing end if the composition is fluid and of a thinner viscosity. If the composition is in a stick format, a container with a propel-repel mechanism wherein the stick is fixed on a platform towards a dispensing orifice is appropriate. If the composition is in an aerosol format, then metallic cans pressurized by a propellant and having a spray nozzle is appropriate. In general, patches, bottles, tubes, roller-ball applicators, squeeze containers or lidded jars are preferred.

For the avoidance of doubt, the term “comprising” is meant not to be limiting to any stated elements but rather to encompass non-specified elements of major or minor functional importance. Therefore, the listed steps, elements or options need not be exhaustive. Whenever the words “including” or “having” are used, these terms are meant to be equivalent to “comprising” as defined above. The term comprises is also meant to encompass the terms consisting essentially of and consisting of. For the avoidance of doubt, a composition comprising resorcinol, a functionalized heteroaromatic compound, and retinoic acid precursor in combination with antioxidant, compatible oil, and low HLB, high molecular weight emulsifier to provide for improved color stability of the composition is meant to include a composition consisting essentially of the same and a composition consisting of the same. As to the percentages used herein, the same are meant to be by weight of ingredient (e.g., not including any water it may be supplied with), unless noted otherwise.

The term “skin” as used herein includes the skin on the face, neck, chest, back, arms, axilla, buttocks, hands, legs and scalp. Skin benefit agent, as used herein, is meant to include a component that improves a facial or body characteristic after topical application like a skin characteristic and/or benefits the same wherein the skin benefit agent can be incorporated in a desirable format. The term “derivative(s)” as used with reference to ingredients herein refers to compounds which may be formed from a precursor compound and/or is a structural or chemical analog. All states of matter, as used herein, such as solid, liquid and gas relate to the specified state of matter at 25° C. and atmospheric pressure. The term “functionalized,” as used herein, means that a compound possesses at least one functional group substituent, including an acyl, hydroxy, alkoxy, carboxamide (amide), carboxyl, or carboalkoxy (ester) substituent, on the compound. Ambient temperature, as used herein, refers to a temperature of 20 to 25° C. When referring to oil solubility and/or water solubility (e.g., “water-soluble” or “oil-soluble), the conditions are ambient temperature and atmospheric pressure.

The following Examples are provided to further illustrate an understanding of the invention. The Examples are not intended to limit the scope of the claims. One of ordinary skill in the art will recognize that variations of the method steps that differ from the examples given may be practiced without deviating from the teachings of the present compositions.

### EXAMPLES

All samples were made by mixing the mentioned ingredients under conditions of moderate sheer, 22° C. to 85° C., and atmospheric pressure. All δₜ data were collected from the CRC Handbook of Solubility Parameters and Other Cohesion Parameters, Second Edition (by Allan F. M Barton).

### Example 1

| | Weight percent (wt %) | | | | | | |
|---|---|---|---|---|---|---|---|
| Ingredients | 1 | 2 | 3 | A | B | 4 | 5 |
| Water | Balance | Balance | Balance | Balance | Balance | Balance | Balance |
| Carbomer (polyacrylate) | 0.4 | — | — | 0.4 | 0.4 | 0.4 | — |
| Potassium hydroxide 45% | 0.1 | — | — | 0.1 | 0.1 | 0.1 | — |
| Humectant | 3.5 | 3.5 | 3.5 | 3.5 | 3.5 | 3.5 | 3.5 |
| Preservative | 0.3 | 0.3 | 0.3 | 0.3 | 0.3 | 0.3 | 0.3 |
| Niacinamide | 3.0 | 3.0 | 3.0 | 3.0 | 3.0 | 3.0 | 3.0 |
| Retinyl propionate | 0.3 | 0.3 | 0.3 | 0.3 | 0.3 | 0.3 | 0.3 |
| 4-hexylresorcinol | 0.4 | 0.4 | 0.4 | 0.4 | 0.4 | 0.4 | 0.4 |
| Isopropyl myristate (δₜ = 17.5) | 10 | 10 | 10 | 10 | — | — | 25 |
| Isodecyl Neopentanoate (δₜ = 15.8) | — | — | — | — | — | 15 | — |
| Mineral Oil (δₜ = 14.5) | — | — | — | — | 15 | — | — |
| Caprylic/capric triglyceride (δₜ = 18.9) | 5.0 | 5.0 | 5.0 | 5.0 | — | — | 5.0 |
| Cetyl Alcohol | 0.5 | 0.5 | 0.5 | 0.5 | 0.5 | 0.5 | 0.5 |
| Pentaerythrityl Tetra-di-t-butyl | 0.1 | 0.1 | — | — | 0.1 | 0.1 | 0.1 |
| Hydroxyhydrocinnamate | | | | | | | |
| Dilauryl Thiodipropionate | 0.1 | 0.1 | — | — | 0.1 | 0.1 | 0.1 |
| Butylated hydroxytoluene (BHT) | — | — | 0.2 | — | — | — | — |
| PEG-30 dipolyhydroxystearate | 2.0 | 2.0 | — | 2.0 | 2.0 | 2.0 | 2.0 |
| (HLB = 5.5) | | | | | | | |
| TOTAL | 100.0 | 100.0 | 100.0 | 100.0 | 100.0 | 100.0 | 100.0 |
| Contains compatible oil | Yes | Yes | Yes | Yes | No | Yes | Yes |
| Contains antioxidant | Yes | Yes | Yes | No | Yes | Yes | Yes |
| Contains low HLB, high molecular | Yes | Yes | Yes | Yes | Yes | Yes | Yes |
| weight, emulsifier | | | | | | | |
| ΔΕ (45° C. at 4 weeks) | 1.72 | 2.98 | 3.72 | 6.01 | 7.74 | 5.10 | 1.90 |
| ΔΕ (45° C. at 8 weeks) | 4.11 | 5.11 | 6.94 | 10.5 | 14.4 | 9.20 | 2.73 |

Example 1 demonstrated that compositions comprising 4-substituted resorcinol (e.g., 4-hexylresorcinol), functionalized heteroaromatic compound (e.g., niacinamide) and a retinoic acid precursor (e.g., retinyl propionate) in a compatible oil carrier with antioxidant are color stable in 45° C. at 4-week and 8-week conditions. Cosmetic compositions were made by combining the above ingredients in accordance with the disclosed cosmetic compositions.

Samples 1 to 5 are in scope of the disclosed cosmetic composition, each comprising a compatible oil, antioxidant, and low HLB, high molecular weight emulsifier. Sample 5 comprises high levels of compatible oil. Resultant ΔE values are below 5.5 at 45° C. at 4 weeks and 9.5 at 45° C. at 8 weeks, showing superior color stability.

Samples A and B are not made according to the disclosed cosmetic composition (i.e., are comparative examples) as Sample A does not contain any antioxidants while Sample B comprises mineral oil, which does not have a Hansen solubility parameter value of 15 to 23. Both Samples A and B result in compositions with ΔE values higher than 5.5 at 45° C. at 4 weeks and 9.5 at 45° C. at 8 weeks.

### Example 2: Varying Impact of Emulsifiers

| | Weight percent (wt %) | | | |
|---|---|---|---|---|
| Ingredients | 1 | C | 6 | 7 |
| Water | Balance | Balance | Balance | Balance |
| Carbomer (polyacrylate) | 0.4 | — | 0.4 | — |
| Potassium hydroxide 45% | 0.1 | — | 0.1 | — |
| Humectant | 3.5 | 3.5 | 3.5 | 3.5 |
| Preservative | 0.3 | 0.3 | 0.3 | 0.3 |
| Niacinamide | 3.0 | 3.0 | 3.0 | 3.0 |
| Retinyl propionate | 0.3 | 0.3 | 0.3 | 0.3 |
| 4-hexylresorcinol | 0.4 | 0.4 | 0.4 | 0.4 |
| Isopropyl myristate (δₜ = 17.5) | 10 | 10 | 7.5 | 7.5 |
| Caprylic/capric triglyceride | 5.0 | 5.0 | 5.0 | 5.0 |
| (δₜ = 18.9) | | | | |
| Cetyl Alcohol | 0.5 | 0.5 | 0.5 | 0.5 |
| Pentaerythrityl Tetra-di-t-butyl | 0.1 | 0.1 | 0.1 | 0.1 |
| Hydroxyhydrocinnamate | | | | |
| Dilauryl Thiodipropionate | 0.1 | 0.1 | 0.1 | 0.1 |
| PEG-30 dipolyhydroxystearate | 2.0 | | | |
| (HLB = 5.5; MW = 2,400 Da) | | | | |
| Glyceryl monostearate | | 2.0 | | |
| (HLB = 4.2; MW = 358.6) | | | | |
| Cetyl PEG/PPG-10/1 | | | 2.0 | |
| Dimethicone (HLB = ~5; | | | | |
| MW = 625.2 Da) | | | | |
| Polyglyceryl-6 Polyricinoleate | | | | 2.0 |
| (HLB = ~3; MW = ~960 Da) | | | | |
| TOTAL | 100.0 | 100.0 | 100.0 | 100.0 |
| Contains compatible oil | Yes | Yes | Yes | Yes |
| Contains antioxidant | Yes | Yes | Yes | Yes |
| Contains low HLB, high | Yes | No | Yes | Yes |
| molecular weight emulsifier | | | | |
| ΔΕ (45° C. at 4 weeks) | 1.72 | Separated | 4.06 | 4.45 |
| | | (not | | |
| | | measurable) | | |
| ΔE (45° C. at 8 weeks) | 4.11 | Separated | 7.73 | 7.37 |
| | | (not | | |
| | | measurable) | | |

Example 2 demonstrated the impact of varying emulsifiers has on the cosmetic compositions disclosed herein. Cosmetic compositions were made by combining the above ingredients in accordance with the disclosed cosmetic compositions. Samples 1, 6, and 7 are in scope of the disclosed cosmetic composition, each comprising a different low HLB, high molecular weight emulsifier. Resultant ΔE values are below 5.5 at 45° C. at 4 weeks and 9.5 at 45° C. at 8 weeks. In contrast, Sample C is not made according to the disclosed cosmetic composition (i.e., is a comparative example) as it comprises glyceryl monostearate, which is a low HLB, low molecular weight emulsifier. Sample C demonstrates that absence of a low HLB, high molecular weight emulsifier results in unstable compositions.

1. A cosmetic composition comprising:
(a) 0.001 to 10% by weight of a resorcinol comprising 4-ethylresorcinol, 4-hexylresorcinol, 4-phenylethylresorcinol, 4-cyclopentyl resorcinol, 4-cyclohexylresorcinol, 4-octylresorcinol, isobutylamido thiazolyl resorcinol (thiamidol), or mixtures thereof;
(b) a retinoic acid precursor comprising, retinol, retinal ester, retinyl ester, or mixtures thereof;
(c) a functionalized heteroaromatic compound comprising nicotinic acid, picolinic acid, nicotinate, niacinamide, picolinamide, or a mixture thereof;
(d) 10 to 40% by weight of an oil where the oil has a Hansen total solubility parameter from 15 to 23;
(e) an antioxidant;
(f) a nonionic emulsifier having an HLB value less than or equal to 8 and having a number average molecular weight greater than 500 Da.
wherein the composition is oil continuous.

2. (canceled)

3. The cosmetic composition according to claim 1, wherein the retinoic acid precursor comprises of retinyl propionate, retinol, or mixtures thereof, and further wherein the composition comprises 0.001 to 10% by weight of the retinoic acid precursor.

4. The cosmetic composition according to claim 1, wherein the composition comprises 0.001 to 10% by weight of the functionalized heteroaromatic compound.

5. The cosmetic composition according to claim 1, wherein the oil has a Hansen total solubility parameter from 16 to 22.

6. The cosmetic composition according to claim 1, wherein the oil makes up from 14 to 25% by weight of the composition.

7. The cosmetic composition according to claim 1, wherein the oil comprises caprylic/capric triglycerides, ethylhexyl salicylate, isodecyl neopentanoate, isopropyl myristate, isopropyl palmitate, octocrylene, octylmethoxycinnamate, or a mixture thereof.

8. The cosmetic composition according to claim 1, wherein the nonionic emulsifier has an HLB value less than or equal to 7.

9. The cosmetic composition according to claim 1, wherein the nonionic emulsifier is present in an amount of 0.1 to 10% by weight of the cosmetic composition.

10. The cosmetic composition according to claim 1, wherein the nonionic emulsifier comprises PEG-30 dipolyhydroxystearate, cetyl PEG/PPG-10/1 dimethicone, polyglyceryl-6 polyricinoleate, polyglyceryl-3 diisostearate, or a mixture thereof.

11. The cosmetic composition according to claim 1, wherein the antioxidant comprises BHT, didodecyl 3,3′ thiodipropionate, octadecyl di-t-butyl-4-hydroxyhydrocinnamate, pentaerythrityl tetra-di-t-butyl hydroxyhydrocinnamate, polyphenols, or a mixture thereof, and wherein the antioxidant is present in a total weight percent of 0.01 to 4% by weight of the cosmetic composition.

12. The cosmetic composition according to claim 1, wherein the composition further comprises at least one of a sunscreen, photostabilizer, skin-lightening agent, wrinkle-reducing agent, coloring agent, retinoid booster, or a mixture thereof, and further wherein the retinoid booster comprises 12-hydroxystearic acid, cetyl alcohol, cetearyl alcohol, cocamide monoethanolamide, linoleoyl monoethanolamide, climbazole, or a mixture thereof.

13. The cosmetic composition according to claim 1, wherein the composition is substantially free of silicones, synthetic polymer microparticles, or both.

14. The cosmetic composition according to claim 1, wherein the composition has a ΔE value of less than or equal to 5.5 at 4 weeks at 45° C.

15. The cosmetic composition according to claim 1, wherein the composition has a ΔE value of less than or equal to 9.5 at 8 weeks at 45° C.

16. The cosmetic composition according to claim 1, wherein the composition comprises 0.01 to 5% resorcinol by weight of the composition.

17. The cosmetic composition according to claim 1, wherein the composition comprises 10 to 40% oil by weight of the composition.

18. The cosmetic composition according to claim 1, wherein the nonionic emulsifier has an HLB less than or equal to 6.

19. The cosmetic composition according to claim 1, wherein the oil has a Hansen total solubility parameter from 17 to 21.

20. The cosmetic composition according to claim 1, wherein the oil makes up from 15 to 20% by weight of the composition.

21. The cosmetic composition according to claim 1, wherein the nonionic emulsifier is present in an amount of 0.5 to 7% by weight of the cosmetic composition.
